(19) United States
(12) Patent Application Publication
(10) Pub. No.: US 2024/0074442 A1
(43) Pub. Date: Mar. 7, 2024

(54) UVARIA GRANDIFLORA EXTRACT AS A BOTANICAL FUNGICIDE, PREPARATION METHOD THEREFOR, AND APPLICATION THEREOF

(71) Applicant: HAINAN UNIVERSITY, HAIKOU (CN)

(72) Inventors: RANFENG SUN, HAIKOU (CN); JIANGUO HE, HAIKOU (CN); ZHAN HU, HAIKOU (CN); JIA XIE, HAIKOU (CN)

(21) Appl. No.: 18/487,399

(22) Filed: Oct. 16, 2023

Related U.S. Application Data
(63) Continuation of application No. PCT/CN2021/122048, filed on Sep. 30, 2021.

(30) Foreign Application Priority Data
Apr. 21, 2021 (CN) 202110430140.2

(51) Int. Cl. A01N 65/08; A01N 37/10

(52) U.S. Cl. CPC A01N 65/08; A01N 37/10

(57) ABSTRACT

The present application provides an Uvaria grandiflora extract as a botanical fungicide, a preparation method therefor, and an application thereof. The broad-spectrum plant-derived fungicide provided in the present application comprises: Uvaria grandiflora whole plant extract and compound zeylenone obtained through separation and purification, which have the advantages of a broad antibacterial spectrum and a simple separation process. By means of a potted fungicidal activity test, it is proven that Uvaria grandiflora extracts have excellent control effects on Phytophthora capsici, cucumber downy mildew, potato late blight, and cowpea powdery mildew. Furthermore, Uvaria grandiflora extracts promote cucumber growth. Furthermore, a field efficacy test proves that the fungicidal activity of Uvaria grandiflora extracts against cucumber powdery mildew is better than that of the commercial fungicide Luna Sensation. The Uvaria grandiflora extracts can be used as a potential plant-derived fungicide for preventing and controlling plant diseases.

### TECHNICAL FIELD

The present application relates to the technical field of fungicides, in particular to an Uvaria grandiflora extract as a botanical fungicide, a preparation method therefor, and an application thereof.

### BACKGROUND ART

With the enhancement of people's awareness of environmental protection, the call for advocating the nature, protecting the environment and paying attention to food safety is growing day by day, and pollution-free biological pesticides, as well as biological control technologies and concepts have gradually attracted great attention. Natural fungicides separated from plants have been successfully used to manage plant diseases, and have advantages over chemically synthesized pesticides, such as containing new compounds that are not yet resistant to known phytopathogenic fungi, relatively fast biodegradation, less residue, renewable materials, and the like. At present, extracts of many plants have been proven to have fungicidal activity against phytopathogenic fungi, such as thyme, garlic, Cnidium monnieri, dandelion, Reynoutria japonica, and the like. However, some of these known natural products feature low fungicidal activity or narrower targets, and do not have broad-spectrum fungicidal activity, but involve complex isolation procedures at a relatively high cost. Therefore, the market is in urgent need of discovering and developing a series of inexpensive and environmentally friendly plant-derived fungicides featuring high fungicidal activity and wide fungicidal range, and the preparation methods therefor.

People mainly use Uvaria grandiflora to treat sore throat. Uvaria grandiflora contains a variety of active compounds including terpenoids, amides, epoxyethylene oxides, β-sitosterol, anthracene, benzoic acid and carotenoids, and the like, among which zeylenone is the most biologically active compound. Studies have shown that zeylenone can inhibit the proliferation of cervical cancer cells and induce the apoptosis through PI3K/AKT/mTOR and MAPK/ERK pathway. Specifically, the patent titled “zeylenone and Derivatives Thereof, Preparation Methods Therefor and Application Thereof” (Application No. CN202011262882.0) discloses that zeylenone and its derivatives have anti-nasopharyngeal carcinoma drugs, anti-human liver cancer drugs, anti-human lung cancer drugs, anti-human glioblastoma drugs, anti-human prostate cancer drugs, and anti-mouse pancreatic cancer; Zeylenone can treat gastric cancer by inducing mitochondrial apoptosis and inhibiting proliferation, migration, and invasion of cells. Through studies of inhibitory effects of zeylenone on prostate cancer, researchers found that zeylenone decreased expression levels of MMP-2, MMP-9, and fibronectin 1 while increasing expression levels of tissue inhibitors of metalloproteinase-1 and collagen-1, and that zeylenone decreased expression levels of wnt5a, β-catenin and cyclin D1, therefore, zeylenone could become a new drug for the treatment of prostate cancer. Through studies of inhibitory effects of zeylenone on ovarian cancer, researchers found that zeylenone was capable of significantly increasing the reduction of mitochondrial membrane potential and apoptosis, not only made the cyto c (cyto c) and apoptosis-inducing factor (AIF) mRNA and protein levels increased, but also drove up the Caspase-3, Fas, Fasl and Bax mRNA and protein levels. Furthermore, zeylenone significantly decreased expression levels of tyrosine kinase p-JAK and signal transducers and activators of transcription (p-STAT), therefore, zeylenone could become a new drug for the treatment of ovarian cancer. Further, the patent titled “zeylenone Derivatives, Preparation Method Therefor and Application Thereof” (Application No.: CN202011282034.6) also shows that zeylenone derivatives can be applied to the preparation of anti-malignant melanoma drugs, anti-human liver cancer drugs, anti-human neuroblastoma drugs, and anti-dog mammary tumors drugs. Scientific experiments have also demonstrated that zeylenone had good anti-cancer effects in the treatment of chronic myelogenous leukemia, which reduced the viability and proliferation of K562 cells in a dose-dependent manner, and induced apoptosis, specifically, zeylenone inhibited the phosphorylation of Jak2 and Src and down-regulated their downstream proteins, including p-Stat3, p-PI3K, p-AKT, p-mTOR, and p-ERK. In a mouse xenograft model of K562 cells, zeylenone also inhibited tumor growth with low toxicity; and further studies have shown that zeylenone has efficient anti-tuberculosis activity. To sum up, a large number of medical experiments have shown that ketenes exhibited high-efficiency biological activities in anti-tumor and anti-tuberculosis, but the activities of the Uvaria grandiflora extract and zeylenone in anti-phytopathogenic fungi have not been reported yet.

### SUMMARY

In view of this, the present application provides the Uvaria grandiflora extract as a botanical fungicide, preparation method therefor, and application thereof, so as to solve the problem of plant diseases.

The technical scheme of the present application is implemented in the following way: Uvaria grandiflora extract as a botanical fungicide, and the fungicide is methanol crude extracts or ethyl acetate extracts of the whole plant of the Uvaria grandiflora.

Preferably, the preparation method for an Uvaria grandiflora extract as a botanical fungicide, including the following steps: taking whole plant of fresh Uvaria grandiflora and cutting into sections, drying the sections at 48-52° C. and then grinding them into powder, weighing the powder and soaking in a methanol solution with a volume fraction of 99-99.5% for 2-4 times for 70-75 h each time, a mass to volume ratio (kg/L) of the Uvaria grandiflora powder to the methanol solution being 2-3:20, combining the soaking solution for filtration and concentrating the filtered solution under vacuum conditions of 40-45° C. to obtain the methanol crude extracts of the whole plant of Uvaria grandiflora.

Preferably, the preparation method for an Uvaria grandiflora extract as a botanical fungicide, including the following steps:

S1. preparing the methanol crude extracts of Uvaria grandiflora: taking whole plant of fresh Uvaria grandiflora and cutting into sections, drying the sections at 48-52° C. and then grinding them into powder, weighing the powder and soaking in a methanol solution with a volume fraction of 99-99.5% for 2-4 times for 70-75 h each time, a mass to volume ratio (kg/L) of the Uvaria grandiflora powder to the methanol solution being 2-3:20, combining the soaking solution for filtration and concentrating the filtered solution under vacuum conditions of 40-50° C. to obtain the methanol crude extracts of Uvaria grandiflora.

S2. preparing the ethyl acetate extracts of Uvaria grandiflora: dissolving the methanol crude extracts obtained in the step S1 in water to obtain an aqueous solution of methanol crude extracts, then extracting the solution with ethyl acetate for 2-4 times to obtain an organic phase, respectively, and concentrating the organic phase under vacuum conditions of 40-50° C. to obtain the ethyl acetate extracts;

S3. performing chromatographic separation: performing column chromatographic separation of the ethyl acetate extracts obtained in the step S2 with petroleum ether and/or ethyl acetate solvent system, and a volume ratio of the petroleum ether to the ethyl acetate being 100:0, 95:5, 90:10, 80:20, 50:50, and 0:100, respectively, to obtain fractions F1, F2, F3, F4, F5, and F6; and

S4. performing elution: taking the fraction F4 and using a silica gel column to perform elution with the eluent being petroleum ether and ethyl acetate solvents, to obtain four fractions labeled as Fr1, Fr2, Fr3, and Fr4, where the Fr4 is a compound zeylenone through purification.

Preferably, a mass volume ratio (kg/L) of the methanol crude extract to the water in the step S2 is 0.80-1.2:5.

Preferably, the water in the step S2 is ultrapure water or distilled water.

Preferably, a volume ratio of the ethyl acetate to the aqueous solution of the methanol crude extract in the step S2 is 1.8-2.2:1.

Preferably, the zeylenone is applied in preparing the extract as a botanical fungicide, and a chemical formula of the zeylenone is C₁₉H₂₉NO₃, as shown in the structural formula I:

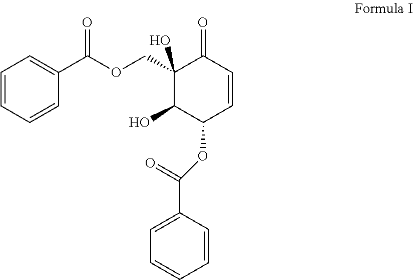 [SMILES: O=C(OC[C@]1(O)C(=O)C=C[C@H](OC(=O)c2ccccc2)[C@H]1O)c1ccccc1]

The present application provides methods for the extraction of crude extracts and separation of active ingredients from the whole plant of Uvaria grandiflora, the separated active compounds are identified, and the crude extracts and zeylenone are then used to carry out in vitro and in vivo fungicidal activity tests on 10 common phytopathogenic fungi and oomycetes by the mycelial growth rate method.

The phytopathogenic fungi specifically include:
Ithizoctonia solani; Pyricularia oryzae; Fusarium graminearum; Colletotrichum siamense; Colletotrichum musae; Colletotrichum gloeosporioiles; Colletotrichum capsici; Phytophthora capsici; Gilbertella persicaria; Neoscytalidium dimidiatum; Podosphaera xanthii; Erysiphe cichoracearum; and Pseudoperonospora cubensis.

Compared with the prior art, the present application has the following beneficial effects:
Uvaria grandiflora whole plant extract and compound zeylenone obtained through separation and purification have the advantages of high efficiency, strong fungicidal activity and a broad fungicidal spectrum. By means of a potted fungicidal activity test, it is proven that Uvaria grandiflora extracts have excellent control effects on Phytophthora capsici, cucumber downy mildew, potato late blight, and cowpea powdery mildew. Furthermore, Uvaria grandiflora extracts promote cucumber growth. A field efficacy test proves that the fungicidal activity of Uvaria grandiflora extracts against cucumber powdery mildew is better than that of the commercial fungicide Luna Sensation. The Uvaria grandiflora extracts can be used as a potential plant-derived fungicide for preventing and controlling plant diseases.

zeylenone extracted and separated from Uvaria grandiflora exhibited broad-spectrum antifungal activity, with the best inhibitory effects on Gloeosporium musarum Cooke et Mass and Phytophthora capsici. The EC50 of zeylenone on Gloeosporium musarum Cooke et Mass and Phytophthora capsici are 3.37 and 6.98 μg mL⁻¹, respectively. Furthermore, by means of a potted fungicidal activity test, it is shown that the control effect of zeylenone on cucumber downy mildew is equivalent to that of the chemical fungicide Cyazofamid, and it could be applied as a lead compound of the botanical fungicide.

### DESCRIPTION OF THE DRAWINGS

FIG. 1 illustrates growth-promoting test of microemulsion of 5% Uvaria grandiflora crude extracts on cucumber.

### DETAILED DESCRIPTION OF THE EMBODIMENTS

In order to have a better understanding of the technical content of the present application, specific embodiments are provided below to give a preferred description of the present application.

Experimental methods used in the following examples of the present application, unless otherwise specified.

Unless otherwise specified, Materials, reagents, and the like, used in the examples of the present application are commercially available, unless otherwise specified.

### Example 1—Preparation of Methanol Crude Extracts from the Whole Plant of Uvaria Grandiflora

Fresh Uvaria grandiflora plants were cut into small sections, put into an air drying oven for drying at 50° C., and then ground into powder. 2.5 kg of the powder was weighed and soaked in 20 L of methanol solution with a volume fraction of 99.5% for 3 times for 72 h each time, the socking solution was combined and filtered, and then concentrated with a rotary evaporator under vacuum conditions of 45° C. to obtain the methanol crude extracts of the whole plant of Uvaria grandiflora.

### Example 2—Preparation of Methanol Crude Extracts from the Whole Plant of Uvaria Grandiflora

Fresh Uvaria grandiflora plants were cut into small sections, put into an air drying oven for drying at 48° C., and then ground into powder. 2 kg of the powder was weighed and soaked in 20 L of methanol solution with a volume fraction of 99% for 2 times for 70 h each time, the socking solution was combined and filtered, and then concentrated with a rotary evaporator under vacuum conditions of 40° C. to obtain the methanol crude extracts of the whole plant of Uvaria grandiflora.

### Example 3—Preparation of Methanol Crude Extracts from the Whole Plant of Uvaria Grandiflora

Fresh Uvaria grandiflora plants were cut into small sections, put into an air drying oven for drying at 52° C., and then ground into powder. 3 kg of the powder was weighed and soaked in 20 L of methanol solution with a volume fraction of 99.5% for 4 times for 75 h each time, the socking solution was combined and filtered, and then concentrated with a rotary evaporator under vacuum conditions of 50° C. to obtain the methanol crude extracts of the whole plant of Uvaria grandiflora.

(1) In Vitro Fungicidal Activity Test

The mycelial growth rate method was adopted to test the fungicidal activity of the methanol crude extracts of Uvaria grandiflora obtained in the above Examples 1-3.

The methanol crude extracts of Uvaria grandiflora obtained in the Examples 1-3 were dissolved in DMF (0.1 mL), respectively, and then mixed with potato dextrose agar (PDA; 9.9 mL), and finally formulated to the desired concentration. The crude extracts were tested for activity at a concentration of 500 μg/mL. All strains were incubated on PDA at 27±1° C. for 4-7 days to ensure that the mycelium had relatively strong activity for identifying the antifungal activity. The test was carried out on a sterile operating table, a bacterium cake with a diameter of about 5 mm was cut from the culture medium covered with mycelium, and the bacterium cake was incubated into the middle of a drug-treated PDA plate with a sterile inoculating needle and cultured at 27±1 for 4-7 days. An equal volume of DMF was added to sterile distilled water as a negative control, and three replicates for each treatment condition in the three examples were performed. The length of fungal colony diameter was measured. The inhibition rate was calculated according to the following formula:

I(%)=((C−T)/(C−0.5))×100
C: the diameter of fungal growth on the PDA of the control group;
T: the diameter of the fungus on the drug-treated PDA;
I: inhibition rate.

TABLE 1
Fungicidal activity of the methanol crude extracts from the whole plant of Uvaria grandiflora against 10
phytopathogenic fungi

| | Concentration | Inhibition rate (%) ±SD | | | | | | | | | |
|---|---|---|---|---|---|---|---|---|---|---|---|
| | (μg mL⁻¹) | R.Sª | P.O | F.G | C.S | C.M | C.G | C.C | P.C | G.P | N.D |
| Example 1 | 500 | 87.3 ± | 81.2 ± | 80.0 ± | 53.0 ± | 92.1 ± | 66.7 ± | 71.3 ± | 94.1 ± | 95.5 ± | 63.3 ± |
| | | 0.5 | 0.8 | 0.2 | 0.5 | 0.6 | 0.2 | 0.7 | 0.2 | 0.4 | 0.9 |
| Example 2 | 500 | 87.1 ± | 81.2 ± | 79.9 ± | 52.3 ± | 92.0 ± | 66.5 ± | 71.2 ± | 94.0 ± | 95.2 ± | 63.2 ± |
| | | 0.5 | 0.8 | 0.2 | 0.5 | 0.6 | 0.2 | 0.4 | 0.1 | 0.5 | 0.8 |
| Example 3 | 500 | 87.2 ± | 81.1 ± | 79.8 ± | 53.0 ± | 91.1 ± | 65.7 ± | 71.1 ± | 94.0 ± | 95.3 ± | 63.1 ± |
| | | 0.5 | 0.8 | 0.2 | 0.5 | 0.5 | 0.6 | 0.5 | 0.2 | 0.4 | 0.9 |
| Osthole | 50 | 75.8 ± | 89.1 ± | 65.1 ± | 37.7 ± | 93.3 ± | 56.9 ± | 4.8 ± | 55.7 ± | 94.3 ± | 68.2 ± |
| | | 0.4 | 0.3 | 0.4 | 0.9 | 0.3 | 0.8 | 0.4 | 0.8 | 0.6 | 0.8 |
| Azoxystrobin | 50 | 73.1 ± | 57.8 ± | 65.7 ± | 60.9 ± | 51.2 ± | 52.5 ± | 41.27 ± | 69.9 ± | 57.2 ± | 51.2 ± |
| | | 0.6 | 0.5 | 0.7 | 0.4 | 0.8 | 0.5 | 0.3 | 0.3 | 0.3 | 1.2 |

ªR.S: Rhizoctonia solani;
P.O: Pyricularia oryzae;
F.G: Fusarium graminearum;
C.S: Colletotrichum siamense;
C.M: Colletotrichum musae;
C.G: Colletotrichum gloeosporioiles;
C.C: Colletotrichum capsici;
P.C: Phytophthora capsici;
G.P: Gilbertella persicaria;
N.D: Neoscytalidium dimidiatum

As can be seen from the above table, the methanol crude extracts from the whole plant of Uvaria grandiflora have better fungicidal activity than that of osthole and azoxystrobin against the above 10 phytopathogenic fungi, and the methanol crude extracts from the whole plant of Uvaria grandiflora obtained in the Example 1 exhibits the best fungicidal activity.

(2) Potted Fungicidal Activity Test

The methanol crude extracts of Uvaria grandiflora obtained in the above Example 1 was prepared as 5% Uvaria grandiflora crude extract microemulsion for determining the inhibitory effects on Phytophthora capsici, cucumber downy mildew, potato late blight, cucumber powdery mildew and cowpea powdery mildew. The Uvaria grandiflora crude extract microemulsion with different concentrations was evenly sprayed on leaf surfaces of pepper, cucumber, potato and cowpea, and after 24 h of treatment, the above pathogenic fungi were inoculated. The inoculated leaves were cultured at a temperature of 25±30° C. and relative humidity of 80±85%. An equal volume of distilled water was used as a negative control, and commercial drugs were used as a positive control, see Table 2 for details. The degree of infection was then calculated and determined according to the disease level of each pathogen, and the protective effect was calculated according to the following formula:

Disease index=Σ(Disease grade×Number of diseased leaves at this grade)/Total number of investigated leaves×Number of the highest grade×100

Prevention and treatment effect (%)=(Disease index of the control group−Disease index of the treatment group)/(Disease index of the control group)×100

TABLE 2
Potted prevention and treatment effect of the microemulsion of 5%
methanol crude extracts from the whole plant of Uvaria grandiflora
and zeylenone on cucumber downy mildew and potato late blight

| | Concentration | Control effect (%) ± SD | |
|---|---|---|---|
| | (μg mL⁻¹) | P.C | P.I |
| Microemulsion of 5% | 500 | 100 ± 0.0 | 100 ± 0.0 |
| methanol crude extracts | 250 | 100 ± 0.0 | 98 ± 1.0 |
| from the whole plant of | 125 | 98 ± 1.0 | 30 ± 2.0 |
| Uvaria grandiflora | 62.5 | 82 ± 2.0 | 0 ± 0.0 |
| | 31.25 | 20 ± 2.0 | 0 ± 0.0 |
| zeylenone | 50 | 99 ± 2.0 | 20 ± 2.0 |
| | 25 | 88 ± 1.7 | 0 ± 0.0 |
| | 12.5 | 70 ± 3.0 | 0 ± 0.0 |
| | 6.25 | 25 ± 1.0 | 0 ± 0.0 |
| Cyazofamid | 25 | 80 ± 2.0 | —^{b} |
| | 12.5 | 70 ± 1.7 | — |
| | 6.25 | 0 ± 2.0 | — |

^{a}P.C: Cucumber downy mildew;
P.I: potato late blight;
^{b}“—” means no data.

TABLE 3
Potted prevention and treatment effect of the
microemulsion of 5% methanol crude extracts from the whole
plant of Uvaria grandiflora on Phytophthora capsici

| | | Protective effect | |
|---|---|---|---|
| | Concentration | Disease index | Control |
| | (μg mL⁻¹) | (±SD) | effect (%) |
| Microemulsion of 5% | 1000 | 0.0 ± 0.0 | 100.0 |
| methanol crude extracts | 500 | 30.0 ± 2.0 | 62.5 |
| from the whole plant of | 250 | 32.0 ± 4.0 | 60.0 |
| Uvaria grandiflora | 125 | 52.0 ± 2.0 | 35.0 |
| Berberine hydrochloride | 200 | 16.0 ± 2.0 | 80.0 |
| Dimethomorph | 200 | 0.0 ± 0.0 | 100.0 |
| Blank control | —^{a} | 80.0 ± 3.5 | — |

^{a}“—” means no data.

TABLE 4
Potted prevention and treatment effect of the microemulsion
of 5% methanol crude extracts from the whole plant of
Uvaria grandiflora on cowpea powdery mildew

| | | 14 days after spraying | |
|---|---|---|---|
| | Concentration | Disease index | Control |
| | (μg mL⁻¹) | (±SD) | effect (%) |
| Microemulsion of 5% | 1000 | 1.5 ± 0.5 | 98.4 |
| methanol crude extracts | 500 | 10.5 ± 0.3 | 89.1 |
| from the whole plant of | 250 | 30.2 ± 0.7 | 68.5 |
| Uvaria grandiflora | | | |
| Propiconazole | 50 | 0 ± 0.0 | 100 |
| Blank control | —^{a} | 95.8 ± 0.2 | — |

^{a}“—” means no data.

TABLE 5
Field prevention and treatment effect of the microemulsion of 5% methanol crude extracts from
the whole plant of Uvaria grandiflora on cucumber powdery mildew

| | | | 6 days after the 1^{st} | | 7 days after the 2^{nd} | |
|---|---|---|---|---|---|---|
| | Use | Disease index | spraying | | spraying | |
| | amount | (±SD) before | Disease | Control | Disease | Control |
| | (g/ha) | spraying | index (±SD) | effect (%) | index (+SD) | effect (%) |
| Microemulsion of 5% | 350^{a} | 14.2 ± 1.2 | 13.0 ± 1.6 | 30.95 | 4.3 ± 1.7 | 80.58 |
| methanol crude extracts | | | | | | |
| from the whole plant of | 175 | 14.2 ± 1.0 | 9.9 ± 1.6 | 47.40 | 4.3 ± 1.0 | 80.63 |
| Uvaria grandiflora | 87.5 | 13.6 ± 1.4 | 9.9 ± 1.9 | 44.95 | 4.3 ± 2.3 | 79.72 |
| Luna Sensation | 200.9^{b} | 24.1 ± 1.7 | 10.5 ± 1.8 | 67.01 | 11.1 ± 1.6 | 70.56 |
| Blank control | _—^{c} | 17.3 ± 1.5 | 22.8 ± 2.8 | — | 27.1 ± 4.5 | |

^{a}refers to the use amount of Uvaria grandiflora methanol crude extracts; ^{b}refers to the use amount of active ingredients; and ^{c}“—” means no data.

TABLE 6
E₅₀ value of fungicidal activity of the
methanol crude extracts from the whole plant of
Uvaria grandiflora against phytopathogenic fungi

| | Pathogenic | |
|---|---|---|
| | fungi | EC₅₀ (μg mL⁻¹) ± SD |
| Uvaria grandiflora | R.S ^{a} | 100.84 ± 0.06 |
| methanol crude extracts | P.O | 300.47 ± 0.00 |
| | F.G | 591.26 ± 1.23 |
| | P.C | 171.39 ± 0.15 |
| | C.M | 59.31 ± 0.08 |
| | G.P | 118.12 ± 1.05 |
| Azoxystrobin | R.S | <0.06 ± 0.12 |
| | P.O | 17.89 ± 0.14 |
| | F.G | 5.27 ± 0.21 |
| | P.C | 18.81 ± 0.31 |
| | C.M | 52.51 ± 0.42 |
| | G.P | 39.09 ± 0.05 |
| Osthole | R.S | 16.02 ± 1.39 |
| | P.O | 11.54 ± 0.42 |
| | F.G | 21.42 ± 0.61 |
| | P.C | 52.59 ± 0.27 |
| | C.M | 9.18 ± 0.33 |
| | G.P | 11.40 ± 0.51 |

^{a} R.S.: Rhizoctonia solani;
P.O: Pyricularia oryzae;
F.G: Fusarium graminearum;
C.M: Colletotrichum musae;
P.C: Phytophthora capsici;
G.P: Gilbertella persicaria

### Example 4-Determining Growth-Promoting Effects of Microemulsion of 5% Methanol Crude Extracts from the Whole Plant of Uvaria grandiflora on Cucumber

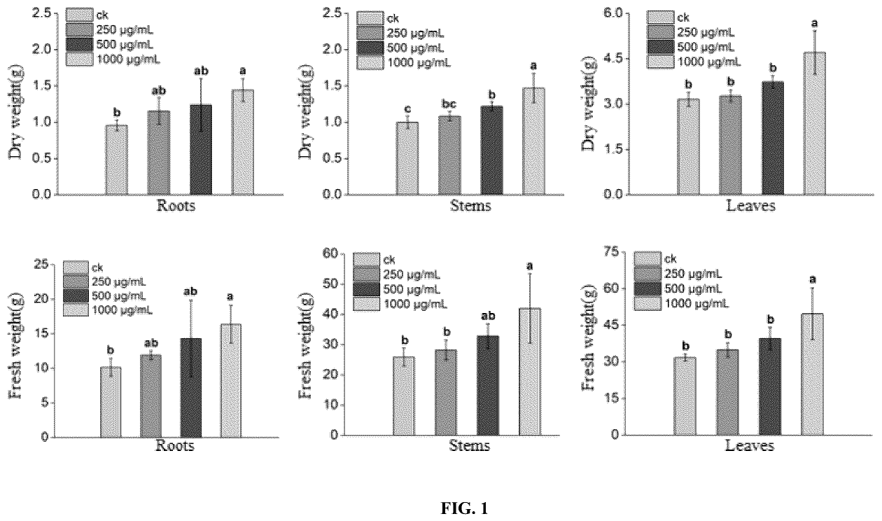

Jinyan No. 4 cucumber seeds purchased from Huayu Company were selected and cultivated in seedling trays. After having two leaves, cucumber seedlings were transplanted to pots, and the cucumber seedlings with uniform growth, uniform size and vigorous vitality were selected as the plants under test for subsequent use, and 3 treatment groups and 1 control groups were arranged for the experiment. The microemulsion of 5% Uvaria grandiflora crude extracts was diluted to 1000 μg/mL, 500 μg/mL, 250 μg/mL, solvent water containing the same amount of solvent was used as blank control, and the solvent was sprayed by using a pressure sprayer, with 50 mL solvent being sprayed for each group. All the tested cucumber plants were placed in a greenhouse incubated at a temperature of 26±1° C. and relative humidity of 70±1%, and the growth-promoting effects of Uvaria grandiflora crude extracts microemulsion on cucumber were evaluated by weighing the fresh and dry weights of cucumber roots, stems, and leaves after 14 days, as shown in Table 7 and FIG. 1.

TABLE 7
Growth-promoting test of microemulsion of 5%
Uvaria grandiflora crude extracts on cucumber

| | | Control | 250 μg/mL | 500 μg/mL | 1000 μg/mL |
|---|---|---|---|---|---|
| Fresh weight | Roots | 10.13 ± 1.30b | 11.88 ± 0.65ab | 14.31 ± 5.53ab | 16.36 ± 2.77a |
| (g) ± SD | Stems | 25.93 ± 2.99b | 28.19 ± 3.29b | 32.81 ± 4.15ab | 41.98 ± 11.52a |
| | Leaves | 31.73 ± 1.39b | 34.93 ± 2.91 b | 39.60 ± 4.59 b | 49.72 ± 10.65a |
| Dry weight | Roots | 0.95 ± 0.08b | 1.16 ± 0.19ab | 1.24 ± 0.36ab | 1.44 ± 0.16a |
| (g) ± SD | Stems | 1.00 ± 0.09c | 1.09 ± 0.07bc | 1.22 ± 0.06b | 1.47 ± 0.20a |
| | Leaves | 3.16 ± 0.24b | 3.27 ± 0.19 b | 3.74 ± 0.20 b | 4.71 ± 0.72a |

P < 0.05

### Example 5—Preparing Zeylenone and Determining the Fungicidal Activity Thereof

(1) Preparing the Uvaria grandiflora Ethyl Acetate Extracts

100 g of methanol crude extracts prepared in the Example 1 were taken and suspended and suspend in 500 mL of ultrapure water to obtain an aqueous solution of methanol crude extracts, the solution was then extracted with ethyl acetate for 3 times to obtain extracted phases, a volume ratio of the ethyl acetate to the aqueous methanol crude extracts for each extraction was 2:1, the three extracted phases were combined and concentrated with a rotary evaporator under vacuum conditions of 50° C., and the ethyl acetate extract was obtained;

(2) Performing Chromatographic Separation

A silica gel column with 200-300 mesh silica gel was used for the initial separation, a petroleum ether/acetic acid solvent system was selected for the column chromatographic separation of the ethyl acetate extracts, and a volume ratio of the petroleum ether to the ethyl acetate being 100:0, 95:5, 90:10, 80:20, 50:50, and 0:100, respectively, to obtain such six fractions F1, F2, F3, F4, F5, and F6;

Performing Elution

Through activity screening, it was identified that the fraction F4 showed the highest fungicidal activity. Therefore, the fraction F4 was taken and eluted with a silica gel column, petroleum ether/ethyl acetate solvent system was used as an eluent for isocratic elution, and four fractions marked as Fr1, Fr2, Fr3, and Fr4 were obtained, and a compound with fungicidal activity, that is, zeylenone, was obtained through activity screening.

(2) Structure Identification

Light yellow powder, mp 154-156° C. (lit. 150-151° C.).

1H NMR (400 MHz, Chloroform-d) δ 8.02 (dd, J=4, 8 Hz, 2H), 7.94 (dd, J=4, 8 Hz, 2H), 7.55 (dd, J=8, 16 Hz, 2H), 7.47-7.36 (m, 4H), 6.96 (ddd, J=10, 4, 1 Hz, 1H), 6.34 (dd, J=10, 1 Hz, 1H), 5.96 (t, 1H), 4.85 (d, J=12 Hz, 1H), 4.60 (d, J=12 Hz, 1H), 4.38 (d, J=5 Hz, 1H), 4.10 (s, 1H), 3.21 (s, 1H).

13C NMR (100 MHz, Chloroform-d) δ 196.2, 166.2, 165.4, 142.7, 133.8, 133.5, 129.8, 129.7, 129.1, 128.8, 128.7, 128.6, 128.5, 77.3, 71.7, 69.2, 65.4.

HRMS (ESI): m/z calcd for C₂₁H₁₉O₇ [M+H]⁺ 383.1125, found: 383.1095.

By analyzing the spectral data, the compound was identified as zeylenone, and the structure thereof is shown in the following Formula I, and the chemical formula is C₁₉H₂₉NO₃.

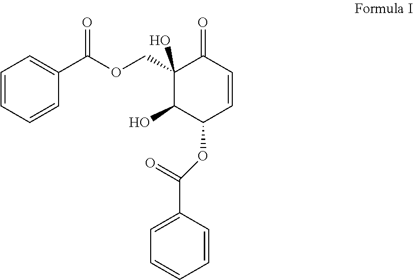 [SMILES: O=C(OC[C@]1(O)C(=O)C=C[C@H](OC(=O)c2ccccc2)[C@H]1O)c1ccccc1]

(3) In Vitro Fungicidal Activity Test

The fungicidal activity of the zeylenone was tested by the mycelial growth rate method.

The zeylenone were taken and dissolved in DMF (0.1 mL), respectively, and then mixed with potato dextrose agar (PDA; 9.9 mL), and finally formulated to the desired concentration, and the fungicidal activity test was performed at a concentration of 50 μg/mL. All strains were incubated on PDA at 27±1° C. for 4-7 days to ensure that the mycelium had relatively strong activity for identifying the antifungal activity. The test was carried out on a sterile operating table, a bacterium cake with a diameter of about 5 mm was cut from the culture medium covered with mycelium, the bacterium cake was incubated into the middle of a drug-treated PDA plate with a sterile inoculating needle and cultured at 27±1 for 4-7 days. An equal volume of DMF was added to sterile distilled water as a negative control, and osthole and azoxystrobin were taken as a positive control, and three replicates for each treatment condition in the three examples were performed. The length of fungal colony diameter was measured. The inhibition rate was calculated according to the following formula:

I(%)=((C−T)/(C−0.5))×100
C: the diameter of fungal growth on the PDA of the control group;
T: the diameter of the fungus on the drug-treated PDA;
I: inhibition rate.

TABLE 8
In vitro fungicidal activity test of zeylenone against phytopathogenic fungi

| | Concentration | Inhibition rate (%) ±SD | | | | | | | | | |
|---|---|---|---|---|---|---|---|---|---|---|---|
| | (μg mL⁻¹) | R.S^{a} | P.C | F.G | C.S | C.M | C.G | C.C | P.C | G.P | N.D |
| Zeylenone | 50 | 76.8 ± | 66.2 ± | 52.5 ± | 43.5 ± | 84.3 ± | 45.0 ± | 34.4 ± | 88.1 ± | 77.6 ± | 43.8 ± |
| | | 0.7 | 0.6 | 0.8 | 0.5 | 0.5 | 1.1 | 0.5 | 0.6 | 0.7 | 0.3 |
| Osthole | 50 | 75.8 ± | 89.1 ± | 65.1 ± | 37.7 ± | 93.3 ± | 56.9 ± | 4.8 ± | 55.7 ± | 94.3 ± | 68.2 ± |
| | | 0.4 | 0.3 | 0.4 | 0.9 | 0.3 | 0.8 | 0.4 | 0.8 | 0.6 | 0.8 |
| Azoxystrobin | 50 | 73.1 ± | 57.8 ± | 65.7 ± | 60.9 ± | 51.2 ± | 52.5 ± | 41.27 ± | 69.9 ± | 57.2 ± | 51.2 ± |
| | | 0.6 | 0.5 | 0.7 | 0.4 | 0.8 | 0.5 | 0.3 | 0.3 | 0.3 | 1.2 |

^{a}R.S: Rhizoctonia solani;
P.O: Pyricularia oryzae;
F.G: Fusarium graminearum;
C.S: Colletotrichum siamense;
C.M: Colletotrichum musae;
C.G: Colletotrichum gloeosporioiles;
C.C: Colletotrichum capsici;
P.C: Phytophthora capsici;
G.P: Gilbertella persicaria;
N.D: Neoscytalidium dimidiatum

TABLE 9
E₅₀ value of zeylenone against phytopathogenic fungi

| | Pathogenic fungi | EC50 (μg mL⁻¹) ± SD |
|---|---|---|
| Zeylenone | R.S | 4.91 ± 0.12 |
| | P.O | 21.38 ± 0.14 |
| | F.G | 22.94 ± 0.11 |
| | P.C | 6.98 ± 0.21 |
| | C.M | 3.37 ± 0.11 |
| | G.P | 17.27 ± 2.29 |
| Azoxystrobin | R.S | <0.06 ± 0.12 |
| | P.O | 17.89 ± 0.14 |
| | F.G | 5.27 ± 0.21 |
| | P.C | 18.81 ± 0.31 |
| | C.M | 52.51 ± 0.42 |
| | G.P | 39.09 ± 0.05 |
| Osthole | R.S | 16.02 ± 1.39 |
| | P.O | 11.54 ± 0.42 |
| | F.G | 21.42 ± 0.61 |
| | P.C | 52.59 ± 0.27 |
| | C.M | 9.18 ± 0.33 |
| | G.P | 11.40 ± 0.51 |

As can be seen from the above table, the Uvaria grandiflora extracts and zeylenone have high and a broad-spectrum fungicidal activity against phytopathogenic fungi.

It can be known from the foregoing examples that the plant extracts and the separated compounds obtained by the present application all have higher fungicidal activity against phytopathogenic fungi under in vitro conditions, and compared with the commercial fungicides (osthole and azoxystrobin), the fungicidal effects of the compound zeylenone have similar or even higher fungicidal effects than those of commercial fungicides and have a broader spectrum.

What is described above is merely preferred examples of the present application, and is not intended to limit the present application. Any modifications, equivalent replacements and improvements, etc. made within the spirit and principle of the present application should fall within the scope of protection of the present application.

What is claimed is:

1. An Uvaria grandiflora extract as a botanical fungicide, wherein the fungicide is methanol crude extracts or ethyl acetate extracts of the whole plant of the Uvaria grandiflora.

2-6. (canceled)

7. A zeylenone is applied in preparing the botanical fungicide, wherein a chemical formula of the zeylenone is C₁₉H₂₉NO₃, as shown in the structural formula I: 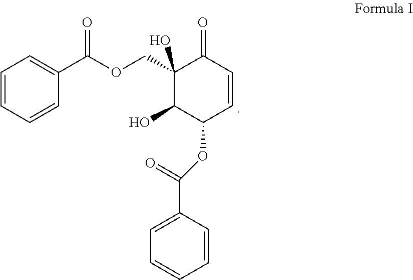 [SMILES: O=C(OC[C@]1(O)C(=O)C=C[C@H](OC(=O)c2ccccc2)[C@H]1O)c1ccccc1]
